(19) United States
(12) Patent Application Publication
(10) Pub. No.: US 2026/0076571 A1
(43) Pub. Date: Mar. 19, 2026

(54) WIRELESS SENSOR READER WITH MULTIPLE FIXED EXCITATION FREQUENCIES

(71) Applicant: ENDOTRONIX, INC., Naperville, IL (US)

(72) Inventors: Balamurugan SUNDARAM, Woodridge, IL (US); Michael NAGY, Lombard, IL (US); Suresh SUNDARAM, Lisle, IL (US)

(21) Appl. No.: 19/396,534

(22) Filed: Nov. 21, 2025

Related U.S. Application Data
(63) Continuation of application No. PCT/US2024/030755, filed on May 23, 2024.
(60) Provisional application No. 63/468,354, filed on May 23, 2023.

(51) Int. Cl. A61B 5/00; A61B 5/0215

(52) U.S. Cl. CPC A61B 5/0031; A61B 5/0215

(57) ABSTRACT

A wireless sensor reader configured to ascertain the resonance frequency of a sensor may comprise a transmit circuit configured to transmit a wireless energizing pulse at a transmit frequency to the sensor, exciting a resonant circuit within the sensor to resonance at a frequency proportional to a measured parameter and a receiver circuit configured to receive a response signal from the sensor, the response signal being a continuous wave at the sensor resonance frequency. The wireless sensor reader may also comprise a circuit for determining the frequency of the sensor response signal, where the reader selects the transmit frequency of the energizing pulse from a plurality of discrete narrowband frequencies, the selection taking place before the determination of the frequency of the sensor response signal.

### CROSS REFERENCE TO RELATED APPLICATIONS

The application is a continuation of International Patent Application No. PCT/US2024/030755 filed May 23, 2024, entitled “WIRELESS SENSOR READER WITH MULTIPLE FIXED EXCITATION FREQUENCIES,” which claims priority to U.S. Provisional Ser. No. 63/468,354 , filed on May 23, 2023, entitled “WIRELESS SENSOR READER WITH MULTIPLE FIXED EXCITATION FREQUENCIES,” each of which is incorporated herein by reference in its entirety.

### TECHNICAL FIELD

This present disclosure relates generally to wireless sensor systems and, more particularly, to wireless sensor systems wherein the sensor is read by a reader, wherein the sensor is an LC resonant tank circuit, and wherein the reader wirelessly excites the sensor to resonance, receives the reflected response signal, and determines its frequency, the frequency having a known relationship to a physical parameter being measured.

### BACKGROUND

Wireless sensor systems may employ resonant circuit technology that utilizes a passive wireless sensor in remote communication with excitation and reader circuitry. Often the wireless sensor is implanted at a specific location, such as within the human body, to detect and report a sensed parameter. In some systems, the sensed parameter varies the resonant frequency of the wireless sensor. A reader may detect the resonant frequency of the wireless sensor to determine the sensed parameter.

In one application, passive wireless sensor systems may employ resonant circuit technology. Passive wireless sensor systems may be pressure monitoring devices, for example, for use by themselves or incorporated into other medical devices including, without limitation, pacemakers, defibrillators, drug elution devices, ventricular assist devices (VADs), and the like. In one embodiment, a medical device may include one or more sensors that are configured to be positioned at a desired location within the human body. The one or more sensors may be fabricated using microelectromechanical systems (MEMS) technology and may be configured to transmit wireless data to an external receiver or reader to facilitate transmission of diagnostic health data to a physician, clinician, a nurse, a patient's caregiver, or the patient, for example.

One such sensor formed using a MEMS technique has an inductive and a capacitive component. For example, the sensor comprises an inductor (L) and a capacitor (C) connected together in parallel, commonly called an LC tank circuit. The geometry of the sensor allows for the deformation of a capacitive plate with increased pressure. This deformation leads to a deflection of the plate and hence causes a change in the capacitance value of the system. The LC tank circuit also generates an electronic resonating frequency. This resonating frequency is related to the inductive and capacitance values of the circuit and will change with the deflection of capacitor plates under changing pressure. This emitted resonating frequency signal is received by an external wireless receiver or reader and deciphered into a correlative pressure reading.

Such sensors may also include wireless data transmission capability. The device may require no battery or internal power. Rather, the sensor may be powered by an inductively coupled electromagnetic (EM) field that is directed towards its inductor coil. The receiver or reader may provide the electromagnetic field by generating a radio frequency (RF) burst or other signal. The sensor's inductor receives energy from the EM field to cause the sensor LC tank to resonate and store energy. When the external EM field is removed, the inductance and capacitance form a parallel resonant circuit to radiate energy through the inductor, which acts as an antenna. This oscillating circuit will then produce RF signals, whose frequency is proportional to the capacitive value of the sensor, which varies with pressure. The sensor's inductor coil may serve both as an inductor creating the oscillating RF signals having a frequency proportional to the capacitance of the sensor at a certain pressure and as an antenna coil emitting the RF signal generated by the LC tank circuitry to provide the oscillating RF signals to the reader.

A pressure sensor may include an inductor/capacitor circuitry assembled in a parallel configuration. In other embodiments, it may include a piezoelectric, piezo-resistive or capacitive pressure sensor. In the inductor/capacitor circuitry, the resonant frequency of the energized circuit will change with the internal pressure of the patient. The sensor transmits sensed or detected pressure readings wirelessly to an external system receiver through RF signals without the requirements of an internal powering system. In a particular embodiment, the sensor may be energized through electromagnetic fields that are directed to a circuitry of the sensor.

Wireless sensor readers intended for frequent use by medical patients at home are particularly useful for taking measurements of internal body parameters of interest to caregivers. In order to ensure patient compliance in taking these readings, consistently, and correctly, however, there is a need for improving the functionality of this system and in particular improving the functionality and usability of the reader. Further, there is a need to allow a user to easily incorporate the reader and associated system within their day-to-day lifestyle and for improving the reliability of the reader in the field, to ensure functionality, accuracy, and secure data management.

Other prior art has proposed swept frequency systems to accomplish the same objective. In these systems, multiple excitation pulses are delivered from the reader to the sensor, each with a different transmit frequency fₓₘₜ and some change in a system parameter is observed, relative to that parameter's value for previous fₓₘₜ values. The changed parameter may be the power delivered to the sensor by the reader, the sensor response or ‘ringback’ signal's amplitude or phase, or some other parameter. When the changed parameter is at or near a maximum or minimum, the sensor is assumed to be at its resonance frequency. The excitation frequency fₓₘₜ that was used to elicit the response at sensor resonance is assumed to match the sensor's resonance frequency, and the physical parameter being measured can now be derived.

A reader application U.S. Pub. No. US20230072070, which is incorporated herein by reference in its entirety, details methods and systems for carrying out this measurement. The methods and systems, however, do not use the swept frequency approach described above. Rather, a single excitation frequency fₓₘₜ is pre-selected before the reading even begins. The excitation frequency excites the sensor to resonance and is then abruptly switched off. The sensor will continue to resonate for a time determined by the fₓₘₜ signal strength, the physical distance (called “link distance”) between the reader and sensor antennas, the RF Quality (Q) factor of the transmitted signal and the sensor, and the delta between fₓₘₜ and the sensor's resonance frequency fᵣₑₛ. As it resonates, the sensor transmits a response or ‘ringback’ signal, which can be received by the reader. The ringback signal can only resonate at fᵣₑₛ, is generally weak and attenuates to zero rapidly. The reader must determine fᵣₑₛ before the response signal vanishes. This “fixed frequency” transmission approach has advantages over the “swept frequency” approach. Its circuitry is generally smaller and lower power, facilitating handheld reader designs. Its control algorithms are generally simpler, and it is less susceptible to false locks on nearby non-sensor radiators or its own sideband frequencies.

The fixed frequency approach, however, has limitations of its own. When the excitation frequency fₓₘₜ is equal to the sensor's resonance frequency fᵣₑₛ, the sensor's resonator stores maximum RF energy, and the response signal it reflects back to the reader is of a high signal strength (SS), providing the reader with a strong signal-to-noise ratio and a longer duration response signal, allowing a more accurate phase or frequency measurement. As fₓₘₜ moves further away from fᵣₑₛ, the sensor's response signal strength weakens quickly. At a certain delta between fₓₘₜ and fᵣₑₛ, the sensor response signal is too weak for the reader to detect. Therefore, a fixed frequency system must be designed so that the reader's energizing signal frequency fₓₘₜ is always near enough to sensor fᵣₑₛ to elicit a signal that is above the minimum signal strength (power amplitude) detection threshold of the reader to provide a more accurate phase or frequency measurement.

Such a design can impose other limitations on the system. The sensor must be designed so that its full scale operating range of fᵣₑₛ is narrow enough to always be within a certain bandwidth, with that bandwidth centered around the reader's fₓₘₜ. For example, a fixed frequency reader with fₓₘₜ =13.5 MHz may be used with a sensor whose operational bandwidth spans 13 to 14 MHz, which corresponds inversely to a pressure measurement range of 550 - 900 mmHg as seen on the x-axis in FIG. 1. The hashed central arrow is the reader's narrowband excitation signal at fₓₘₜ=13.5 MHz. The bell-shaped curve represents the sensor's transfer function, which indicates strength of the sensor's response to that excitation signal at different frequencies. When measured pressure is 737 mmHg, the sensor resonates at fᵣₑₛ=13.462 MHz, as seen at the solid arrow just left of the center arrow. This arrow's magnitude, as dictated by the sensor's response curve, is high, indicating a strong response signal that is well above the reader detection threshold, shown as a hashed horizontal line across the graph. In another measurement, where pressure is 689 mmHg, fᵣₑₛ becomes 13.602 MHz, located to the right of center, somewhat further away from fₓₘₜ, this signal may be slightly less accurate but is still strong enough to be detected. In the case where pressure is far from center, for example 870 mmHg, fᵣₑₛ=13.120 MHz and is too weak for the reader to detect.

The exemplary fixed frequency system of FIG. 1 has a limited operational measurement range, confined to the space between the two points where the sensor response curve intersects the reader detection threshold line, in this example approximately 673 - 778 mmHg. Design of LC resonant tank microsensors with wider operational range typically incurs undesired penalties in size, precision, accuracy, and manufacturability. There is thus a need for a wireless LC resonant tank sensor-reader system that preserves the advantages of a fixed fₓₘₜ architecture yet allows a detection of sensor frequency fᵣₑₛ over wide operating bandwidth.

### SUMMARY

The following presents a summary of this disclosure to provide a basic understanding of some aspects. This summary is intended to neither identify key or critical elements nor define any limitations of embodiments or claims. Furthermore, this summary may provide a simplified overview of some aspects that may be described in greater detail in other portions of this disclosure. Any of the described aspects may be isolated or combined with other described aspects without limitation to the same effect as if they had been described separately and in every possible combination explicitly.

Disclosed is a wireless sensor reader configured to ascertain the resonance frequency of a sensor may comprise a transmit circuit configured to transmit a wireless energizing pulse at a transmit frequency to the sensor, exciting a resonant circuit within the sensor to resonance at a frequency proportional to a measured parameter and a receiver circuit configured to receive a response signal from the sensor, the response signal being a continuous wave at the sensor resonance frequency. The wireless sensor reader may also comprise a circuit for determining the frequency of the sensor response signal, where the reader selects the transmit frequency of the energizing pulse from a plurality of discrete narrowband frequencies, the selection taking place before the determination of the frequency of the sensor response signal, wherein the reader obtains a plurality of samples of the sensor response signal over a measurement time interval, the frequency of each of the plurality of samples of the sensor response signal is determined by the circuit for determining frequency and each of the plurality of samples of the sensor response signal is initiated by an energizing pulse at the same the transmit frequency throughout the measurement time interval.

The described devices, systems, and methods may provide LC resonant tank sensors and microsensors with wider operational range and without undesired penalties in size, precision, accuracy, and manufacturability. The described devices, systems, and methods may provide a wireless LC resonant tank sensor-reader system that preserves the advantages of a fixed fₓₘₜ architecture yet allows a detection of sensor frequency fᵣₑₛ over wide operating bandwidth.

Disclosed is a wireless sensor reader configured to ascertain the resonance frequency of a sensor. In an embodiment, the reader may comprise a transmit circuit configured to transmit a wireless energizing pulse at a transmit frequency to said sensor, exciting a resonant circuit within said sensor to resonance at a frequency proportional to a measured parameter. In an embodiment, the reader may comprise a receiver circuit configured to receive a response signal from said sensor, said response signal being a continuous wave at the sensor resonance frequency. In an embodiment, the reader may comprise a circuit for determining the frequency of said sensor response signal.

In an embodiment, said reader may select said transmit frequency of said energizing pulse from a plurality of discrete narrowband frequencies. In an embodiment, the selection may take place before said determination of said frequency of said sensor response signal. In an embodiment, said reader may obtain a plurality of samples of said sensor response signal over a measurement time interval. In an embodiment, the frequency of each of said plurality of samples of said sensor response signal may be determined by said circuit for determining frequency. In an embodiment, each of said plurality of samples of said sensor response signal may be initiated by an energizing pulse at the same said transmit frequency throughout said measurement time interval.

In an embodiment, the circuit for determining the frequency of said sensor response signal may comprise a phase locked loop configured to lock an internal continuous wave signal to said response signal prior to extinction of said response signal, such that the frequency of said internal signal matches the frequency of said sensor response signal. In an embodiment, said phase locked loop may be further configured to hold said internal signal at a constant frequency prior to the extinction of said response signal. In an embodiment, said constant frequency of said internal signal may be equal to said frequency of said sensor response signal. In an embodiment, said reader may further comprises circuitry for determining the frequency of said held internal signal while it is held at a constant frequency. In an embodiment, said circuitry for determining said frequency of said held internal signal may be configured to measure elapsed time of one period of said internal signal.

In an embodiment, said selection of said transmit frequency of said energizing pulse may take place before said determination of said frequency of said sensor's response signal. In an embodiment, said reader may be further configured to use previously measured data to select an initial said transmit frequency of said energizing pulse at a beginning of said measurement time interval. In an embodiment, said previously measured data may be selected from: ambient pressure, past measurements of mean gauge pulmonary artery pressure, or a calculation that uses these parameters.

In an embodiment, said reader may be further configured to measure signal strength of said sensor response signal. In an embodiment, said reader may be further configured to determine whether said signal strength measurement is within a preset threshold window. In an embodiment, said window may have a lower limit that defines a minimum signal strength needed for frequency detection by said reader, and an upper limit that defines a maximum signal strength allowable to prevent saturation of said receiver circuit. In an embodiment, said reader may be further configured to provide audible, visual, or haptic cues to a user indicating whether or not said sensor response signal strength is within said window. In an embodiment, said reader may be further configured to select said transmit frequency by transmitting a plurality of excitation pulses, each with a different frequency selected from said plurality of said discrete narrowband frequencies and selecting said transmit frequency based on the signal strength of the sensor response signal for each of said excitation pulses.

In an embodiment, said reader may be further configured to repeat a portion of its transmit frequency selection process when more than one of said plurality of discrete narrowband frequencies causes a sensor response signal that saturates said receiver circuit. In an embodiment, said repeated portion may include repetition of a step wherein said user repositions said reader in response to said haptic cues. In an embodiment, said reader may be further configured to use said previously measured data to preclude selection of a transmit frequency from among said plurality of discrete narrowband frequencies, when said transmit frequency cannot be the frequency closest to that of said sensor resonance frequency. In an embodiment, said reader may log data during each reading interval to comprise said previously measured data in future reading intervals.

In an embodiment, said reader may be further configured to analyze said samples of said sensor response signal over a portion of said measurement time interval, to determine whether said frequency of said samples has moved closer to a different one of said plurality of said discrete narrowband frequencies than a current excitation signal frequency. In an embodiment, said reader may be further configured to change said current excitation signal frequency to said different one of said plurality of said discrete narrowband frequencies for a remainder of said measurement time interval. In an embodiment, said plurality of discrete narrowband frequencies may be spaced along a frequency spectrum that spans a full scale range of said sensor resonance frequency.

In an embodiment, a sensor's transfer functions for neighboring said discrete narrowband frequencies on said frequency spectrum may overlap one another to ensure that said sensor response signal at every frequency in said full scale range of said sensor resonance frequency has at least one reader transmit frequency capable of exciting said sensor to provide a sensor response signal of sufficient energy for said reader to determine said response signal's frequency. In an embodiment, said reader may be further configured to select an excitation signal transmit frequency from among said neighboring frequencies when a strength of said response signal is equal for two neighboring frequencies. In an embodiment, said selection of said excitation signal transmit frequency may be carried out by an algorithm selected from a group comprising: selecting transmit frequency closest to estimated sensor resonance frequency based on a measured parameter; selecting transmit frequency based on past readings of the sensor resonance frequency; selecting transmit frequency closest to the center of the full-scale range; selecting the transmit frequency used most often in past readings; selecting the transmit frequency based on data from a medical history of a patient; and selecting the transmit frequency based on a patient's posture, as measured by a tilt sensor on said reader.

In an embodiment, said plurality of samples of said sensor response signal over a measurement time signal may comprise an output waveform of the measured parameter. In an embodiment, said plurality of samples of said sensor response signal may be processed to obtain an output parameter, said sample processing selected from among the following: averaging, lowpass filtering, bandpass filtering, weighted averaging, rolling window average, Fourier transformation, wavelet transformation, differentiation, integration, curve fitting, area under curve calculation, trend analysis, correlation with other datasets, standard deviation, analysis of variance, minimum and maximum detection, rise and fall times, or other mathematical data processes. In an embodiment, said sensor resonance frequency may be proportional to a cardiac pressure, and said sample processing is further selected from among the following: heartrate detection, respiration rate detection, systolic peak detection, diastolic minimum detection, cardiac output estimation, flow rate estimation, detection of arrhythmia, detection of irregular respiration, estimation of compliance of a blood vessel, patient posture, patient activity, patient health status, and comparison of any of these said parameters against predetermined thresholds. In an embodiment, said patient health status may include one or more of vital signs, comorbidities, medications, age, and weight.

In an embodiment, said reader may be further configured to calculate a rate of change trend of said sensor resonance frequency during said measurement time interval, and to automatically switch said excitation transmit frequency to a new value if said rate of change trend indicates that said sensor resonance frequency is likely to remain closer to said new value for a remaining portion of said time interval. In an embodiment, said sensor resonance frequency may be proportional to a cardiac pressure. In an embodiment, said automatic switching of said excitation frequency may occur each time said cardiac pressure approaches a systolic maximum or diastolic minimum. In an embodiment, said circuit for determining frequency may make its determination independent of said transmit frequency of said energizing pulse.

The following description and the drawings disclose various illustrative aspects. Some improvements and novel aspects may be expressly identified, while others may be apparent from the description and drawings.

### BRIEF DESCRIPTION OF THE DRAWINGS

Objects and advantages together with the operation of the invention may be better understood by reference to the following detailed description taken in connection with the following illustrations, wherein:

FIG. 1 illustrates a chart depicting a sensor response curve of the prior art intersected with a reader detection threshold line;

FIG. 2 illustrates a chart depicting a fixed four band transmission and sensor response curves identifying frequency and pressure;

FIG. 3 illustrates a flow chart of fixed frequency transmit control algorithm;

FIG. 4 is an embodiment of a wireless sensor system.

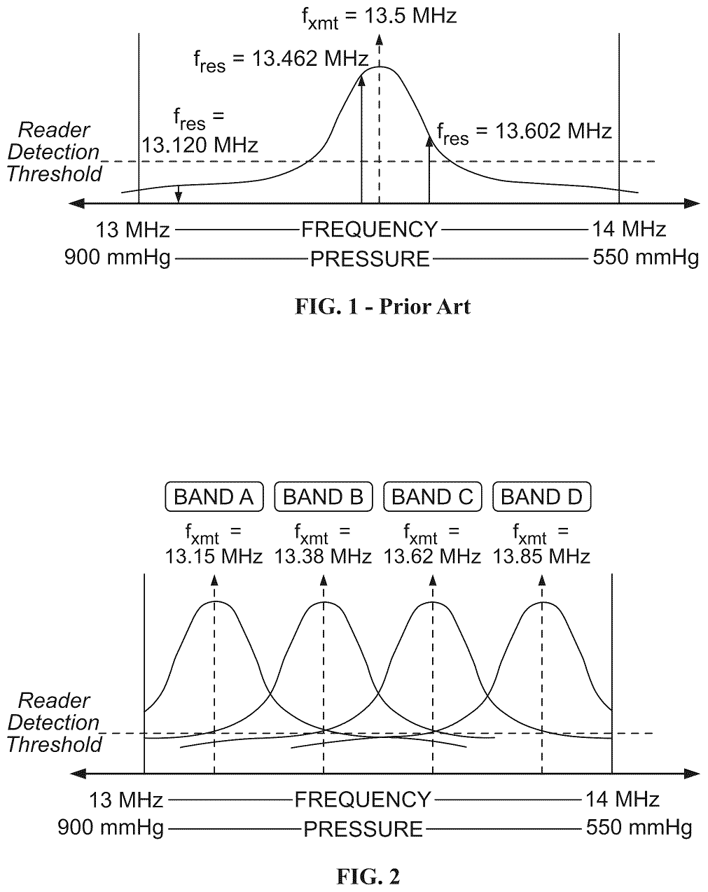

The invention may be embodied in several forms without departing from its spirit or essential characteristics. The scope of the invention is defined in the appended claims, rather than in the specific description preceding them. All embodiments that fall within the meaning and range of equivalency of the claims are therefore intended to be embraced by the claims.

### DETAILED DESCRIPTION

Reference will now be made in detail to exemplary embodiments of the present teachings, examples of which are illustrated in the accompanying drawings, wherein like numbered aspects refer to a common feature throughout. It is to be understood that other embodiments may be utilized and structural and functional changes may be made without departing from the respective scope of the present teachings. Moreover, features of the various embodiments may be combined or altered without departing from the scope of the present teachings. As such, the following description is presented by way of illustration only and should not limit in any way the various alternatives and modifications that may be made to the illustrated embodiments and still be within the spirit and scope of the present teachings.

In this disclosure, numerous specific details provide a thorough understanding of the subject disclosure. It should be understood that aspects of this disclosure may be practiced with other embodiments not necessarily including all aspects described herein.

As used herein, the words “example” and “exemplary” means an instance, or illustration. The words “example” or “exemplary” do not indicate a key or preferred aspect or embodiment. The word “or” is intended to be inclusive rather than exclusive, unless context suggests otherwise. As an example, the phrase “A employs B or C,” includes any inclusive permutation (e.g., A employs B; A employs C; or A employs both B and C). As another matter, the articles “a” and “an” are generally intended to mean “one or more”unless context suggest otherwise.

Disclosed is a wireless sensor reader. In an embodiment, the wireless sensor reader may be configured to ascertain the resonance frequency of a sensor and may comprise a transmit circuit configured to transmit a wireless energizing pulse at a transmit frequency to the sensor, exciting a resonant circuit within the sensor to resonance at a frequency proportional to a measured parameter and a receiver circuit configured to receive a response signal from the sensor, the response signal being a continuous wave at the sensor resonance frequency.

The wireless sensor reader may also comprise a circuit for determining the frequency of the sensor response signal, where the reader selects the transmit frequency of the energizing pulse from a plurality of discrete narrowband frequencies, the selection taking place before the determination of the frequency of the sensor response signal, wherein the reader obtains a plurality of samples of the sensor response signal over a measurement time interval, the frequency of each of the plurality of samples of the sensor response signal is determined by the circuit for determining frequency and each of the plurality of samples of the sensor response signal is initiated by an energizing pulse at the same the transmit frequency throughout the measurement time interval.

Wireless sensor systems may generally include a reader unit or device configured to be placed in a use condition for taking readings from the sensor and in a resting condition in which it is not communicating with the sensor. For example, the disclosed reader may be handheld or battery operated and be adapted for use to operate a few minutes each day. For example, the disclosed reader may be handheld or battery operated and be adapted for use to operate during a discrete or pre-set period of time, including, for example, during a pre-determined physical activity like exercise, walking, biking, and the like. For example, the disclosed reader may be handheld or battery operated and be adapted for use to operate during exercise and the reader may be programmed to dynamically change Tx bands as when necessary across the walk test (e.g., a 9-minute walk test). The disclosed reader may also be configured to sit on a recharging or docking station during times of non-use. It is noted that the disclosed sensor and reader systems may incorporate many types of wireless technology, including, for example, active and passive sensors, continuous wave (CW) and modulated data transmission, analog and digital type systems, and the like.

FIG. 2 illustrates the operational concept of the present system, using exemplary parameter values. Here the reader is designed to transmit excitation pulses at a plurality of discrete, narrowband fixed frequencies, four in this example. It is noted that any number of transmission bands may be used, including two, three, four, five, six, seven, eight, nine, ten, or more transmission bands and fₓₘₜ frequencies. In an example, the reader may transmit more than one, more than two, multiple, or a plurality of transmission bands and fₓₘₜ frequencies. Each of the four fₓₘₜ frequencies shown in FIG. 2 is at the center of a spectral ‘band’ of frequencies, the bands labeled A through D. The sensor's fᵣₑₛ depends on the value of the parameter being measured (pressure in this example), and can be anywhere on the 13-14 MHz spectrum shown, or in other embodiments may be on an alternative spectrum, e.g., 15-16 MHz, 11-12 MHz, 12-13 MHz or 14-15 MHz. The present reader can now select the Band with the fₓₘₜ value that will be closest to fᵣₑₛ, optimizing energy transfer to the sensor during excitation and providing a strong, long-lasting ringback signal to facilitate fᵣₑₛ measurement which can be used in determining pressure in the location of the sensor, such as in the pulmonary artery.

From FIG. 2, it can be seen that every frequency in the sensor's full scale range spectrum has at least one band whose transfer function is above the reader detection threshold. As shown, the neighboring bands overlap, providing a region halfway between two neighboring transmit frequencies where either fₓₘₜ will excite the sensor to produce equal or nearly equal ringback response strength. In actual field use, factors such as environmental noise, reader position, manufacturing tolerances, etc., may cause asymmetry, such that one of the overlapping bands provides better signal strength on the sensor return signal than the other.

In cases where returned signal strength (SS) between neighboring bands is equal or nearly equal, the system will follow an algorithm to select one or the other band and its corresponding fₓₘₜ. Such algorithms may comprise:
Select the fₓₘₜ closest to the estimated fᵣₑₛ that is based on P_{amb}, with or without the mean pulmonary artery pressure (mPAP) offset from the historical readings;
Select the band closest to center of the total bandwidth; and
Select the band based on historical data (the one used more often in the past).

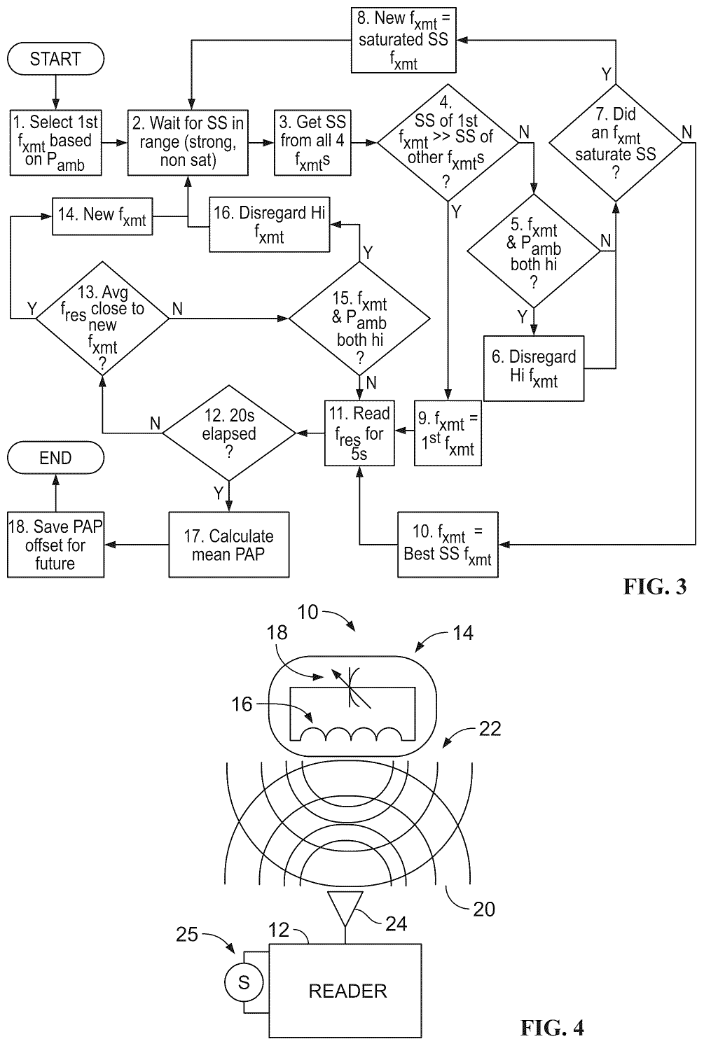

FIG. 3 graphically illustrates the algorithm the reader uses to determine the optimum fₓₘₜ to select for a given reading, from the discrete number of frequencies available. The example illustrated in FIG. 3 is that of a 20 second reading of an LC resonant tank pressure sensor located in the pulmonary artery of a patient. The details of the application and the parametric values provided are exemplary only, and other values may be substituted without altering the present system.

In step 1, at upper left or “start,” the reader selects the first of its discrete transmit frequencies fₓₘₜ to use for sensor excitation. In general, it selects this value based on a rough estimate of the sensor's current fᵣₑₛ. For the specific case exemplified, the reader measures ambient air pressure P_{amb} in its surrounding environment, adds a small amount (typically 5-50 mmHg) to it to account for pulmonary artery pressure imposed by the body, and converts this pressure to frequency based on a pressure-to-frequency calibration lookup table or formula in its memory. The table or formula may be specific to individual sensors or may be generalized to all sensors of that type. Because the patient's pulmonary artery typically adds a small offset to the present atmospheric station pressure, this method can provide a starting point to find the optimum fₓₘₜ from those available. The general concept of using a measured parameter such as P_{amb} to adapt the reader to measurement is detailed in U.S. Pat. No. 8,570,186, which is incorporated herein by reference in its entirety.

In step 2, the first selected fₓₘₜ based on ambient pressure is transmitted and the signal strength (SS), that is the amplitude of the implant's ringback signal, is measured. Signal strength should be a high value, but should not saturate the receiving amplifier circuit used to measure it, as one cannot compare the strengths of different signals if they are both saturated. To bring signal strength (SS) into the desired strength range, the patient may be required to move the handheld reader's position (or, in the case of a pillow-type reader, move their body relative to the reader) in response to audible or visual signals from the reader. For example, the reader may issue a series of audible tones that change volume, pitch, or pulse frequency as the reader gets closer to or further from the location of optimum SS. A different sound may issue when an acceptable SS is achieved. This may take several seconds. Alternatively, a self-adjusting gain amplifier, which could be a logarithmic or other type of amplifier circuit may be used. In the present example, the SS value is converted to a digital code between 0 and 4096, with 3800-4000 considered acceptable SS for home mode, or 3200-4000 in clinic mode.

If all other parameters, such as link distance, antenna tilt angle, frequency-dependent circuit parameters, electrical properties of intervening media, etc., are held constant, the sensor's return signal will always have a larger SS value when its fᵣₑₛ is closer to fₓₘₜ.

Once SS is in range for the first fₓₘₜ, the patient is instructed to not move the reader relative to the body. The system moves on to step 3, and with the reader held in position, SS readings are taken using excitation signals from all fₓₘₜ values (four in the FIG. 3 example). Typically, these SS measurements are done quickly, within 5-20 ms, to finish before the patient moves their hand.

Step 4 determines whether the first fₓₘₜ provides the highest SS. If it does, the rest of the reading process continues using the first fₓₘₜ (step 9).

If it does not, the system proceeds to steps 5 and 6, which perform a ‘sanity check’ on the reading in the case where the highest fₓₘₜ excitation signal (band D in FIG. 2) provides the highest signal strength. With fₓₘₜ in band D, we expect ambient pressure P_{amb} to assume a relatively low value, since measured pulmonary artery pressure is the sum of P_{amb} plus a smaller (typically 5-50 mmHg) pressure added by the body. Because the reader provides a direct measurement of P_{amb}, the system can assess whether P_{amb} is low enough to reasonably expect band D to provide the best SS. If, for example, the reader's on-board pressure sensor measures P_{amb} of more than 700 mmHg, this would correspond to an expected 705-750 mmHg of measured pulmonary artery pressure. In the present example 705-750 mmHg corresponds to fᵣₑₛ of 13.429-13.557 MHz. Inspection of FIG. 2 reveals that this frequency range will provide the strongest SS when fₓₘₜ is selected from band B or band C, and not band D. Should the reader determine that P_{amb}>700 mmHg and the band D fₓₘₜ provides the strongest SS, the measurement is clearly faulty and the system disregards the band D fₓₘₜ and selects the fₓₘₜ from the band with the next highest SS before proceeding with step 7. Note that in this example, applying this concept to the other direction (band A has highest SS when P_{amb} is very low) may not be desirable, as some patients with conditions such as pulmonary hypertension, may have very high pulmonary pressures up to 300 mmHg. In such cases, band A could possibly have the best SS even with a low P_{amb}.

In the example steps above, the expected frequency value corresponds to a fixed assumption that the body adds 5-50 mmHg to ambient pressure. In alternative embodiments, a different assumption could be made based on learned values from past pulmonary artery pressure measurements from that patient, using a weighted or other type of learning algorithm. The concept of using past data to inform assumptions about future measurements can be applied to any type of measurement and is not limited to pulmonary artery pressure. In a specific example embodiment, the reader may log gauge pulmonary pressure (pressure above ambient) for a given patient each day. A rolling window average, for example over the previous 5 days, could then be added to P_{amb} in step 1. The sum of this average and P_{amb} is then converted to frequency, providing an estimate the current fᵣₑₛ. The reader then selects the fₓₘₜ closest to that estimate for the first excitation pulse.

After the ‘sanity check’ of step 6, one proceeds to step 7. Here the reader considers the case where at least one of the four SS values measured in step 3 saturates the receiving circuit. If it did, one can select one of the fₓₘₜ values that caused saturation in step 8 and return to step 2. The audible cues from the reader again guide the patient to find optimal reader position, this time with the new fₓₘₜ, and the process repeats. If more than one fₓₘₜ excitation pulse caused saturation in step 3, step 7 selects the one closest to the frequency corresponding to P_{amb} and repeats the cycle. Eventually, step 7 or step 9 will determine the excitation fₓₘₜ that provides the ringback signal with highest SS.

With fₓₘₜ now selected from the path leading to either step 9 or step 10, the present system can proceed with the reading itself. In step 11, the reader issues excitation pulses at the selected fₓₘₜ for a portion of the total read interval, in this example 5 seconds, and uses the methods described in the reference prior art to determine fᵣₑₛ of the sensor. In a typical embodiment, the reader stimulates the sensor with an excitation pulse 1000 times per second, each time directly sampling the sensor's frequency. One embodiment does this by using a phase locked loop (PLL) to lock an internal reader signal to the received sensor response signal. The reader opens the PLL and holds its output signal's frequency steady after a preset time interval chosen to be slightly shorter than the anticipated duration of the rapidly decaying sensor response signal. With the PLL held steady at a frequency that matches the now-vanished sensor signal, the reader has time to determine that frequency using a zero-crossing timer or other methods known in the art. This and other embodiments are described in detail in the referenced prior art.

In an embodiment, the excitation signal transmit frequency may be carried out by an algorithm selected from a group comprising: selecting transmit frequency closest to estimated sensor resonance frequency based on a measured parameter; selecting transmit frequency based on past readings of the sensor resonance frequency; selecting transmit frequency closest to the center of the full-scale range; selecting the transmit frequency used most often in past readings; selecting the transmit frequency based on data from the medical history of the patient; selecting the transmit frequency based on the patient's posture, as measured by a tilt sensor on said reader. For example, a patient's medical record may indicate that they typically have a PAP of a certain value above ambient. The reader may use this information to determine a likely starting fₓₘₜ for that patient. It is well known in the art that a pateint's PAP typically increases by an amount, typically around 5-15 mmHg, when the patient lies recumbent compared to their PAP when in an upright (sitting or standing) position. The reader may include an accelerometer or other tilt sensor to determine the patient's posture and use this information as an input to prediction of the optimum fₓₘₜ. For example, a starting fₓₘₜ may be derived from ambient pressure plus the patient's typical body-generated PAP, plus the offset due to patient posture as measured for that patient during a clinical calibration visit in which the patient uses the reader to measure PAP delta between recumbent and upright positions.

In an embodiment, the sensor resonance frequency may be proportional to a cardiac pressure, and the sample processing may be further selected from among the following: heartrate detection, respiration rate detection, systolic peak detection, diastolic minimum detection, cardiac output estimation, flow rate estimation, detection of arrhythmia, detection of irregular respiration, estimation of compliance of a blood vessel, patient posture, patient activity, patient health status, and comparison of any of these said parameters against predetermined thresholds. In an example, patient activity may be measured by an on-board accelerometer that determines step rate or number of steps. In an example, health status may include vital signs, comorbidities, medications, age, weight, etc.

In an embodiment, the circuit for determining frequency makes its determination independent of said transmit frequency of said energizing pulse. In an embodiment, the reader does not need to “know” what the energizing signal's transmit frequency was in order to determine the sensor's resonance frequency.

Returning to FIG. 3, step 12 is simply a check to see whether the full time interval for the reading has elapsed. In our pulmonary artery pressure example, this interval is 20 seconds long to allow several respiratory cycles to take place. Throughout the interval, the reader provides audio cues to the patient to hold the device steadily in position against the thorax. During the interval, each fᵣₑₛ sample may be accompanied by its own SS reading, and individual SS readings below a selected threshold may rejected. The measured fᵣₑₛ may be lowpass filtered using a rolling window weighted average or other filtering means. Many other means of lowpass filtering and spurious signal rejection may be employed to attain the most accurate fᵣₑₛ reading.

If the interval has not elapsed, the system can proceed to step 13. This step checks for the case in which the average measured fᵣₑₛ has moved from one transmit band to another during the previous 5-second interval. The reader maximum and minimum fᵣₑₛ from that interval and selects the midpoint. If the midpoint has moved closer to a different fₓₘₜ, the reader changes to that new value in step 14, and returns to step 2, where it may signal the patient to reposition the reader to bring SS into range. Not shown in the figures, the reader may limit the number of times it changes fₓₘₜ in this way in a given measurement interval, to avoid excessive reading time and repositioning for the user. Also, other means besides the midpoint between minimum and maximum fᵣₑₛ may be employed to distill the full pressure waveform over the measurement interval to a single value. Examples include taking the average, the mean, the mode, or a weighted average.

If, from step 13, the filtered measured fᵣₑₛ has not moved closer to a different fₓₘₜ from the one being used currently, the system continues to step 15, which is a repeat of the ‘sanity check’ from step 5. If the system fails the sanity check by selecting an fₓₘₜ that corresponds to a pressure value far lower than ambient, it disregards all fₓₘₜ values at or above that frequency in step 16 and returns to step 2. In FIG. 3, step 15 indicates that fₓₘₜ is compared to P_{amb} as in step 5, but in alternative embodiments it may be compared to the measured fᵣₑₛ midpoint that was calculated in step 13. If the fₓₘₜ is still at a reasonable value and passes the sanity test of step 15, the process returns to step 11 for another measurement interval of exemplary value 5 seconds.

After the measurement interval, the system determines in step 12 whether the complete measurement time (20 seconds in FIG. 3) has elapsed. If it has not, control proceeds to step 13. If it has, the excitation pulses stop and no more fᵣₑₛ samples are taken. The reader's processor calculates the mean pulmonary artery pressure (mPAP) for the entire 20 second reading in step 17.Not shown in the flowchart, the processor may also count the number of fᵣₑₛ samples rejected throughout the process, either for insufficient SS, reading out of an expected range, or for any other reason. If the cumulative number of rejected samples over the time interval exceeds a preset threshold, for example 10% of samples, then the reader may reject the entire reading, return to step 1, and start over. Alternatively, it may issue a message to the user that the reading failed, and wait until a new reading is initiated or advise the user to take action such as contacting customer support.

In step 18, the reader calculates the gauge mPAP, which is the mPAP above P_{amb}. It stores this value and may use it along with other historical values to predict the likely fᵣₑₛ at the beginning of the next reading. Machine learning and Artificial Intelligence algorithms known in the art may be employed to carry out this prediction.

Although FIG. 3 contains parameters specific to the measurement of pulmonary artery pressure (PAP), it can readily be generalized to any wireless sensor measurement based on a passive LS resonance tank that is energized by an outside reader. To accomplish this generalization, one has merely to transform the specific parameters in FIG. 3 to general ones as follows:
P_{amb} can be any measured parameter
The ‘sanity check’ of steps 5 and 15 can be for any combination of parameter values that are not physically likely to occur in a given application; for example in applications where the system reduces pressure below ambient we would reject cases where fₓₘₜ and P_{amb} are both low rather than high
In step 17, other parameters besides the mean of the final parameter may be calculated
In steps 17 and 18 the sensed parameter can be any parameter besides PAP
Steps 17 and 18 are optional, depending on the application
Specific numerical quantities (four fₓₘₜ, 20 s read interval, 5 s subinterval, etc.) can be replaced by any value

Other embodiments of the System carrying out the steps in FIG. 3 may include the following variations:
The system can skip some steps, do them in a different order, repeat or add more
P_{amb} may be any sensed parameter; some other known value for starting value estimation and sanity checking may be available to help the system select its energizing frequency
The fₓₘₜ intervals do not have to be evenly spaced as in FIG. 2; spacing could be different for different patients or readings
All time intervals in the examples can be changed
The reassessment of fₓₘₜ after the 5-second interval may be omitted. In other words, step 11 of FIG. 3 may cover the full reading interval and would proceed to step 17. Steps 12, 13,14, 15 and 16 may be omitted.

An embodiment of a reader may continuously monitor midpoint or average of PAP and determine the trend over time. It may use this trend to predict the optimal time for transitioning to a new band, and it may make the fₓₘₜ transition at an optimum time, ideally before there is a loss of signal strength.

An embodiment of a reader may monitor pulse pressure (PAPmax−PAPmin) for each heartbeat and may predict when the next min or max will occur. If pulse pressure is high enough to cross over between neighboring bands, the algorithm uses the rate of change of pressure with respect to time (dP/dt) to predict the optimum point at which to change fₓₘₜ from one band to the next, and back again. The reader may change bands twice during every heartbeat to optimize fₓₘₜ for the bands nearest the systolic (PAPmax) and the diastolic (PAPmin) frequencies for that implant and patient. In other embodiments, the reader may dither the fₓₘₜ rapidly between the two bands, and averaging the outputs to obtain quasistatic PAP readings. The dithering may be done by alternating between the two fₓₘₜ values, one value for each sample. Or it may take place by alternating the two values within each single sample's excitation burst.

Turning to FIG. 4, shown is an embodiment of a wireless sensor system that may be used with the present disclosure. A wireless system 10 is generally provided. The wireless system 10 may include a wireless reader 12 and a wireless sensor 14. The wireless sensor 14 may be a passive device, such as a device comprising a capacitor 16 and an inductor 18, or an active device. The wireless sensor 14 may be implantable, such as implantable into a living being. For example, the wireless sensor 14 may be implanted in a human body to monitor a condition or parameter within the human body.

The reader 12 may be configured to transmit an excitation pulse 20 to excite the sensor 14. The excitation pulse 20 may cause the sensor 14 to ring or emit a ring signal 22 at its resonant frequency. The resonant frequency of the sensor 14 may vary based on a parameter sensed by the sensor 14. The reader 12 may measure the frequency of the ring signal 22 and determine the sensed parameter. For example, the reader 12 may utilize a formula, lookup table or calibration table to determine the sensed parameter.

The reader 12 may include a receiver to receive the ring signal 22 from the sensor 14. The receiver may comprise an antenna 24 or any other signal receiving device. The receiver may further include one or more filters, such as for example analog or digital filters, to filter the signal 22 received from the sensor 14. The filters may be tuned to a passband to allow a desired frequency bandwidth to be received by the reader 12. In an embodiment, reader may include a sensor 25.

It will be appreciated that the systems and methods set forth herein may be applied to any measured or sensed parameter, such as pressure, temperature, or any other parameter.

The embodiments of the disclosure have been described above and, obviously, modifications and alterations will occur to others upon reading and understanding this specification. The claims as follows are intended to include all modifications and alterations insofar as they are within the scope of the claims or the equivalent thereof.

What is claimed is:

1. A wireless sensor reader configured to ascertain the resonance frequency of a sensor, comprising:
a transmit circuit configured to transmit a wireless energizing pulse at a transmit frequency to said sensor, exciting a resonant circuit within said sensor to resonance at a frequency proportional to a measured parameter;
a receiver circuit configured to receive a response signal from said sensor, said response signal being a continuous wave at the sensor resonance frequency; and
a circuit for determining the frequency of said sensor response signal;
wherein said reader selects said transmit frequency of said energizing pulse from a plurality of discrete narrowband frequencies, said selection taking place before said determination of said frequency of said sensor response signal;
wherein said reader obtains a plurality of samples of said sensor response signal over a measurement time interval;
wherein the frequency of each of said plurality of samples of said sensor response signal is determined by said circuit for determining frequency; and
wherein each of said plurality of samples of said sensor response signal is initiated by an energizing pulse at the same said transmit frequency throughout said measurement time interval.

2. The wireless sensor reader of claim 1, wherein the circuit for determining the frequency of said sensor response signal comprises a phase locked loop configured to lock an internal continuous wave signal to said response signal prior to extinction of said response signal, such that the frequency of said internal signal matches the frequency of said sensor response signal.

3. The wireless sensor reader of claim 2, wherein said phase locked loop is further configured to hold said internal signal at a constant frequency prior to the extinction of said response signal, said constant frequency of said internal signal being equal to said frequency of said sensor response signal.

4. The wireless sensor reader of claim 3, wherein said reader further comprises circuitry for determining the frequency of said held internal signal while it is held at a constant frequency.

5. The wireless sensor reader of claim 4, wherein said circuitry for determining said frequency of said held internal signal is configured to measure elapsed time of one period of said internal signal.

6. The wireless sensor reader of claim 1, wherein said selection of said transmit frequency of said energizing pulse takes place before said determination of said frequency of said sensor's response signal.

7. The wireless sensor reader of claim 1, wherein said reader is further configured to use previously measured data to select an initial said transmit frequency of said energizing pulse at a beginning of said measurement time interval.

8. The wireless sensor reader of claim 7, wherein said previously measured data is selected from: ambient pressure, past measurements of mean gauge pulmonary artery pressure, or a calculation that uses these parameters.

9. The wireless sensor reader of claim 1, wherein said reader is further configured to measure signal strength of said sensor response signal.

10. The wireless sensor reader of claim 9, wherein said reader is further configured to determine whether said signal strength measurement is within a preset threshold window, said window having a lower limit that defines a minimum signal strength needed for frequency detection by said reader, and an upper limit that defines a maximum signal strength allowable to prevent saturation of said receiver circuit.

11. The wireless sensor reader of claim 10, wherein said reader is further configured to provide audible, visual, or haptic cues to a user indicating whether or not said sensor response signal strength is within said window.

12. The wireless sensor reader of claim 11, wherein said reader is further configured to select said transmit frequency by transmitting a plurality of excitation pulses, each with a different frequency selected from said plurality of said discrete narrowband frequencies and selecting said transmit frequency based on the signal strength of the sensor response signal for each of said excitation pulses.

13. The wireless sensor reader of claim 12, wherein said reader is further configured to repeat a portion of its transmit frequency selection process when more than one of said plurality of discrete narrowband frequencies causes a sensor response signal that saturates said receiver circuit, said repeated portion including repetition of a step wherein said user repositions said reader in response to said haptic cues.

14. The wireless sensor reader of claim 7, wherein said reader is further configured to use said previously measured data to preclude selection of a transmit frequency from among said plurality of discrete narrowband frequencies, when said transmit frequency cannot be the frequency closest to that of said sensor resonance frequency.

15. The wireless sensor reader of claim 7, wherein said reader logs data during each reading interval to comprise said previously measured data in future reading intervals.

16. The wireless sensor reader of claim 1, wherein said reader is further configured to analyze said samples of said sensor response signal over a portion of said measurement time interval, to determine whether said frequency of said samples has moved closer to a different one of said plurality of said discrete narrowband frequencies than a current excitation signal frequency, and wherein said reader is further configured to change said current excitation signal frequency to said different one of said plurality of said discrete narrowband frequencies for a remainder of said measurement time interval.

17. The wireless sensor reader of claim 1, wherein said plurality of discrete narrowband frequencies are spaced along a frequency spectrum that spans a full scale range of said sensor resonance frequency.

18. The wireless sensor reader of claim 17, wherein a sensor's transfer functions for neighboring said discrete narrowband frequencies on said frequency spectrum overlap one another to ensure that said sensor response signal at every frequency in said full scale range of said sensor resonance frequency has at least one reader transmit frequency capable of exciting said sensor to provide a sensor response signal of sufficient energy for said reader to determine said response signal's frequency.

19. The wireless sensor reader of claim 18, wherein said reader is further configured to select an excitation signal transmit frequency from among said neighboring frequencies when a strength of said response signal is equal for two neighboring frequencies.

20. The wireless sensor reader of claim 19, wherein said selection of said excitation signal transmit frequency is carried out by an algorithm selected from a group comprising: selecting transmit frequency closest to estimated sensor resonance frequency based on a measured parameter; selecting transmit frequency based on past readings of the sensor resonance frequency; selecting transmit frequency closest to the center of the full-scale range; selecting the transmit frequency used most often in past readings; selecting the transmit frequency based on data from a medical history of a patient; and selecting the transmit frequency based on a patient's posture, as measured by a tilt sensor on said reader.

21. The wireless sensor reader of claim 1, wherein said plurality of samples of said sensor response signal over a measurement time signal comprise an output waveform of the measured parameter.

22. The wireless sensor reader of claim 1, wherein said plurality of samples of said sensor response signal are processed to obtain an output parameter, said sample processing selected from among the following: averaging, lowpass filtering, bandpass filtering, weighted averaging, rolling window average, Fourier transformation, wavelet transformation, differentiation, integration, curve fitting, area under curve calculation, trend analysis, correlation with other datasets, standard deviation, analysis of variance, minimum and maximum detection, rise and fall times, or other mathematical data processes.

23. The wireless sensor reader of claim 22, wherein said sensor resonance frequency is proportional to a cardiac pressure, and said sample processing is further selected from among the following: heartrate detection, respiration rate detection, systolic peak detection, diastolic minimum detection, cardiac output estimation, flow rate estimation, detection of arrhythmia, detection of irregular respiration, estimation of compliance of a blood vessel, patient posture, patient activity, patient health status, and comparison of any of these said parameters against predetermined thresholds.

24. The wireless sensor reader of claim 22, wherein said patient health status includes one or more of vital signs, comorbidities, medications, age, and weight.

25. The wireless sensor reader of claim 1, wherein said reader is further configured to calculate a rate of change trend of said sensor resonance frequency during said measurement time interval, and to automatically switch said excitation transmit frequency to a new value if said rate of change trend indicates that said sensor resonance frequency is likely to remain closer to said new value for a remaining portion of said time interval.

26. The wireless sensor reader of claim 25, wherein said sensor resonance frequency is proportional to a cardiac pressure, and said automatic switching of said excitation frequency occurs each time said cardiac pressure approaches a systolic maximum or diastolic minimum.

27. The wireless sensor reader of claim 1, wherein said circuit for determining frequency makes its determination independent of said transmit frequency of said energizing pulse.

28. A system comprising:
a sensor configured to be implanted into a patient;
a sensor reader comprising:
a transmit circuit configured to excite a resonant circuit within the sensor to resonance at a frequency proportional to a measured parameter;
a receiver circuit receiving a continuous wave at the frequency; and
a circuit that determines a response frequency of said resonance, wherein the sensor reader selects the frequency from a plurality of discrete narrowband frequencies and wherein the selection takes place before determination of the response frequency;
wherein the sensor reader obtains a plurality of samples of the continuous wave over a measurement time interval;
wherein a frequency of each of the plurality of samples of the continuous wave is determined by the circuit for determining frequency; and
wherein each of the plurality of samples is initiated by an energizing pulse at a same transmit frequency throughout the measurement time interval.

29. A method to ascertain a resonance frequency of a sensor, the method comprising:
transmitting a wireless energizing pulse at a transmit frequency to the sensor via a sensor reader;
exciting a resonant circuit within said sensor to resonance at a frequency proportional to a measured parameter;
receiving a response signal from said sensor, said response signal being a continuous wave at the sensor resonance frequency; and
determining the frequency of said sensor response signal, wherein the sensor reader selects the transmit frequency of the energizing pulse from a plurality of discrete narrowband frequencies, the selection taking place before determination of the frequency of the sensor response signal;
obtaining a plurality of samples of the sensor response signal over a measurement time interval, wherein the frequency of each of the plurality of samples of the sensor response signal is determined by a circuit of the sensor reader for determining frequency; and
wherein each of the plurality of samples of the sensor response signal is initiated by an energizing pulse at a same frequency as the transmit frequency throughout the measurement time interval.
